(19) United States
(12) Patent Application Publication
(10) Pub. No.: US 2017/0197004 A1
(43) Pub. Date: Jul. 13, 2017

(54) APPARATUS AND METHOD FOR GENERATING AROMATIC SMELL IN ELECTRONIC DEVICE

(71) Applicant: Samsung Electronics Co., Ltd., Gyeonggi-do (KR)

(72) Inventor: Gesik KIM, Gyeonggi-do (KR)

(73) Assignee: Samsung Electronics Co., Ltd.

(21) Appl. No.: 15/403,728

(22) Filed: Jan. 11, 2017

(30) Foreign Application Priority Data
Jan. 12, 2016 (KR) 10-2016-0003567

(51) Int. Cl. A61L 9/03

(52) U.S. Cl. CPC A61L 9/035; A61L 2209/133

(57) ABSTRACT

An aromatic smell generation apparatus of an electronic device is provided. The aromatic smell generation apparatus includes a protection case including a cover on one side that protects a display of the electronic device and an electronic device mounting portion on an other side in which the electronic device is located, a cartridge attachment portion to detachably receive different types of a plurality of aromatic smell generation cartridges, and a controller that controls a type and a diffusion time of a perfume stick within the plurality of aromatic smell generation cartridge according to a preset event.

### PRIORITY

This application claims priority under 35 U.S.C. §119(a) to Korean Patent Application Serial No. 10-2016-0003567 which was filed on Jan. 12, 2016 in the Korean Intellectual Property Office, the entire disclosure of which is incorporated herein by reference.

### BACKGROUND

1. Field of the Disclosure

The present disclosure generally relates to an apparatus and method for generating an aromatic smell in an electronic device or accessory, and more particularly, to an apparatus and method whereby such generated aromatic smell corresponds to an event such as a phone call, an alarm occurrence, or a user defined event of the electronic device.

2. Description of the Related Art

With the development of the electronic communication industry, electronic devices have become an important means of information transfer, and electronic devices such as a mobile communication terminal (smart phone), electronic scheduler, personal digital assistant (PDA), television (TV), and laptop computer are widely used. In general, such electronic devices enable a user to conveniently perform work through a graphical user interface (GUI) environment using a touch screen and provide various multimedia based on Internet access and a web environment.

Electronic devices have been developed with the gradual addition of various functions, thus, various accessories have appeared.

For example, an electronic device case may be used as a means for safely protecting an electronic device from drop damage, may be shaped into various forms according to an electronic device kind, and be produced using a soft or hard material of various synthetic resin materials or leather materials.

Further, several products that generate an aromatic smell using a case or a ring can be attached to the electronic device by attaching and detaching an aroma container that generates aromatic smell, or that houses aroma by attaching a sticker type pad containing aroma.

However, because a conventional perfume stick attached to a case or a ring of the electronic device has a structure that performs only an aroma function by simply housing liquid aroma and that continuously generates aromatic smell, the perfume stick may not satisfy the desires of users.

Accordingly, attempts have been made to house in the case or the ring various aromas on a color basis and generate desired aromatic smells, but do not satisfy user expectations.

Further, an attempt has been made to satisfy user expectations by detachably attaching various aromatic smell modules to an electronic device case in order to spray an aromatic smell when desired by a user, but configuring an aromatic smell module in a spray type causes complexity problems and the spray configuration cost is increased.

### SUMMARY

In accordance with an aspect of the present disclosure, an apparatus and method are provided for generating aromatic smell that enables users to use an electronic device with a pleasant feeling by programming the apparatus to generate various aromatic smells according to an event such as an incoming call, a message, a user defined event, and an alarm occurring in the electronic device.

Accordingly, an aspect of the present disclosure provides an aromatic smell generation apparatus of an electronic device which includes a protection case comprising a cover of one side that protects a display of the electronic device and an electronic device mounting portion of the opposite side in which the electronic device is attached or located, a cartridge attachment portion detachably attached to receive different types of a plurality of aromatic smell generation cartridges in the cover, and a controller that controls a type and a diffusion time of a perfume stick within the aromatic smell generation cartridge according to a preset event from the mobile terminal.

Another aspect of the present disclosure provides a method of generating aromatic smell in an electronic device, which includes attaching the electronic device to a protection case that attaches an aromatic smell generation apparatus that generates aromatic smell from a plurality of aromatic smell generation cartridges that each house different types of perfume sticks, authenticating, by the electronic device, the electronic device attached to the protection case, setting an event in the electronic device, and generating an aromatic smell of perfume sticks of at least one aromatic smell generation cartridge of the protection case according to the preset event, when the electronic device detects an event in the protection case.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a top plan view illustrating an aromatic smell generation apparatus of an electronic device, according to an embodiment of the present disclosure;

FIG. 2 is a top plan view illustrating a mobile terminal attached to the case of FIG. 1, according to an embodiment of the present disclosure;

FIG. 3 is a cross-sectional view illustrating the aromatic smell generation apparatus taken along line A-A of FIG. 1, according to an embodiment of the present disclosure;

FIG. 4 is a flowchart of a method of generating aromatic smell in an electronic device according to an embodiment of the present disclosure; and

FIG. 5 is a diagram illustrating a method of generating aromatic smell in an electronic device according to an embodiment of the present disclosure.

### DETAILED DESCRIPTION

Hereinafter, the present disclosure will be described with reference to the accompanying drawings. Although specific embodiments are illustrated in the drawings and related detailed descriptions are disclosed, the present disclosure may have various modifications and several embodiments. However, various embodiments of the present disclosure are not limited to a specific implementation form and it should be understood that the present disclosure includes all changes and/or equivalents and substitutes included in the spirit and scope of of the present disclosure. In connection with descriptions of the drawings, similar components may be designated by the same reference numeral.

Expressions such as “include” and “may include” which may be used in the present disclosure refer to the presence of the disclosed functions, operations, and constituent elements and do not limit one or more additional functions, operations, and constituent elements. In the present disclosure, terms such as “include” and/or “have” may refer to denote a certain characteristic, number, step, operation, constituent element, component or a combination thereof, but may not be construed to exclude the existence of or a possibility of addition of one or more other characteristics, numbers, steps, operations, constituent elements, components or combinations thereof.

In an embodiment of the present disclosure, the expressions “or” or “at least one of A or/and B” include any or all of combinations of words listed together. For example, the expressions “A or B” or “at least A or/and B” may include A, may include B, or may include both A and B.

The expressions “1st”, “2nd”, “first”, or “second” as used in of the present disclosure may modify various components of the embodiments, but do not limit the corresponding components. For example, the above expressions do not limit the sequence and/or importance of the components. The expressions may be used for distinguishing one component from other components. For example, a first user device and a second user device indicate different user devices although both of them are user devices. For example, without departing from the scope of the present disclosure, a first structural element may be referred to as a second structural element. Similarly, the second structural element also may be referred to as the first structural element.

The expression “configured to” as used in the present disclosure may be used interchangeably, according to situations, with “suitable for”, “having the capacity to”, “designed to”, “adapted to”, “made to”, or “capable of”. The term “configured to” may not always mean “specially designed to”. In some situations, “device configured to” may mean that the device may “do something” with other devices or components. For example, a context “processor configured to execute A, B, and C” may mean a dedicated processor (for example, an embedded processor) for executing a corresponding operation, or a general-purpose processor (for example, CPU or application processor) capable of executing corresponding operations by using at least one software program stored in a memory device.

The terms used in the present disclosure are used to describe a specific embodiment, and do not limit the scope of other embodiments. A singular form may include a plural form. All the terms including a technical or scientific term may have the same meaning as terms generally understood by those skilled in the relevant art. The terms defined in a general dictionary may be interpreted as having the same or similar meaning in a context of related technology, and are not to be interpreted abnormally or excessively unless clearly defined in the present disclosure. According to situations, the terms defined in the present disclosure cannot be interpreted as excluding the embodiments of the present disclosure.

An electronic device according to an embodiment of the present disclosure may be a device including a communication function. For example, the electronic device may be one or a combination of a smart phone, a tablet personal computer (PC), a mobile phone, a video phone, an e-book reader, a desktop PC, a laptop PC, a netbook computer, a personal digital assistant (PDA), a camera, a wearable device (for example, a head-mounted-device (HMD) such as electronic eyeglasses, electronic clothes, an electronic bracelet, an electronic necklace, an electronic appcessary, an electronic tattoo, and a smart watch).

According to an embodiment of the present disclosure, the electronic device may be a smart home appliance. The smart home appliance may include at least one of a television (TV), a digital video disk (DVD) player, an audio player, an air conditioner, a cleaner, an oven, a microwave oven, a washing machine, an air cleaner, a set-top box, a TV box (for example, Samsung HomeSync™, Apple TV™, or Google TV™), game consoles, an electronic dictionary, an electronic key, a camcorder, and an electronic frame.

According to an embodiment of the present disclosure, the electronic device may include at least one of various types of medical devices (for example, magnetic resonance angiography (MRA), magnetic resonance imaging (MRI), computed tomography (CT), a scanner, an ultrasonic device and the like), a navigation device, a global positioning system (GPS) receiver, an event data recorder (EDR), a flight data recorder (FDR), a vehicle infotainment device, electronic equipment for a ship (for example, a navigation device for ship, a gyro compass and the like), avionics, a security device, a head unit for a vehicle, an industrial or home robot, an automatic teller machine (ATM), and a point of sale (POS) terminal.

According to an embodiment of the present disclosure, the electronic device may include at least one of furniture or a part of a building/structure, an electronic board, an electronic signature receiving device, a projector, and various types of measuring devices (for example, a water meter, an electricity meter, a gas meter, a radio wave meter, and the like). The electronic device may be one or a combination of the above described various devices. Further, the electronic device may be a flexible device. It is apparent to those skilled in the art that the electronic device is not limited to the above described devices.

The embodiments disclosed in this specification and drawings are presented to describe technical contents of the present disclosure and help the understanding of the present disclosure and do not limit the scope of the present disclosure. Therefore, all changes or modifications derived from the technical idea of the present disclosure as well as the embodiments described herein should be interpreted to belong the scope of the present disclosure.

An electronic device having a waterproof structure for a display module according to an embodiment of the present disclosure may include a short range communication module for short range communication. As a short range communication technique, Bluetooth, radio frequency identification (RFID), infrared data association (IrDA), ultra wideband (UWB), ZigBee, near field communication (NFC), and the like may be used.

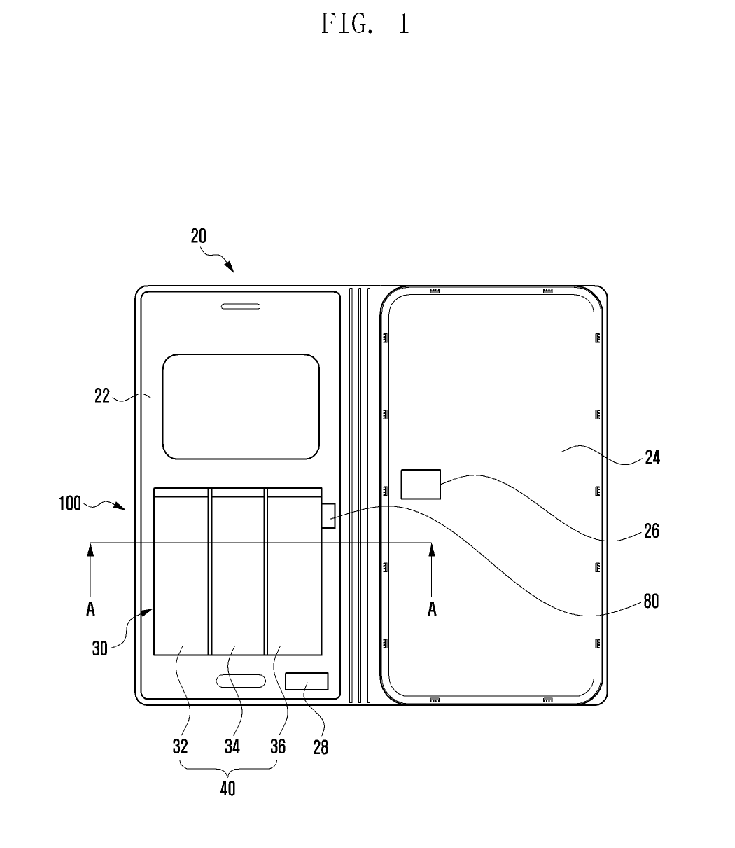

FIG. 1 is a top plan view illustrating an aromatic smell generation apparatus of an electronic device according to an embodiment of the present disclosure.

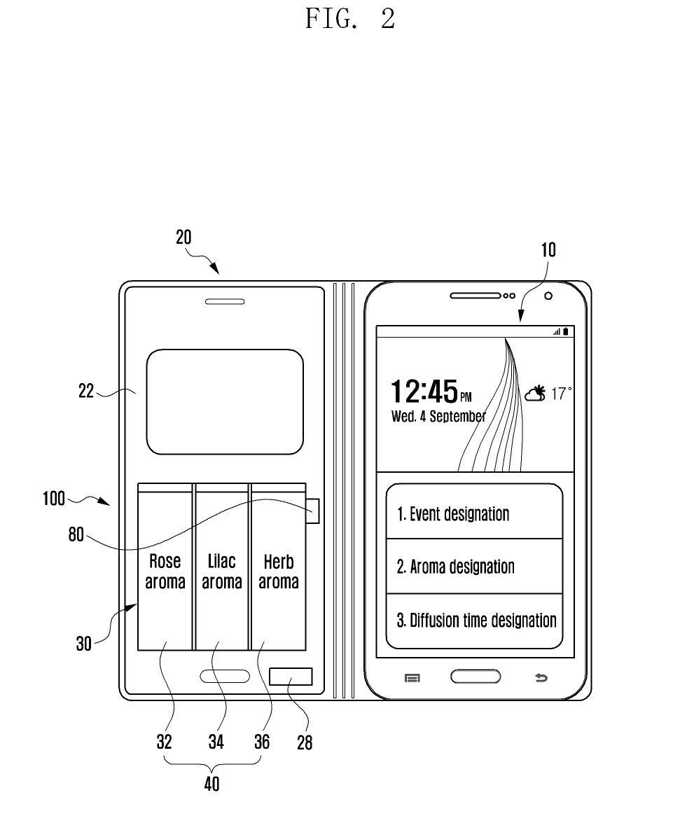

FIG. 2 is a top plan view illustrating a mobile terminal attached to a case of FIG. 1 according to an embodiment of the present disclosure.

Referring to FIGS. 1 and 2, the electronic device may be a mobile terminal 10 and various accessories such as a mobile phone ring, case, touch pen, auxiliary battery, solar power or wireless charge cradle, and waterproof case may be attached to such a mobile terminal 10. An aromatic smell generation apparatus 100 according to an embodiment of the present disclosure may be attached to a protection case 20, which is one of the accessories.

The protection case 20 of the mobile terminal 10 includes a mobile terminal mounting portion 24 in which one side of an inner surface is a cover 22 that protects a display of the mobile terminal 10 and in which the mobile terminal 10 is attached or located at the opposite side thereof.

The cover 22 includes a cartridge attachment portion 30 to attach a receiving case 40 that may detachably receive different types of a plurality of aromatic smell generation cartridges 32, 34, and 36.

The mobile terminal mounting portion 24 includes a near field communication integrated circuit (NFC IC) 26 having an NFC reader function in order to determine that the mobile terminal 10 is covered by the cover 22 in a state in which the mobile terminal 10 is attached, and the cover 22 may include a magnet 28 that determines detachment and attachment of the mobile terminal 10. When the mobile terminal 10 is mounted in the mobile terminal mounting portion 24 of the protection case 20, it may be recognized with NFC authentication of the mobile terminal 10 through the NFC IC 26 that the cover 22 is attached by the magnet 28 of the cover 22.

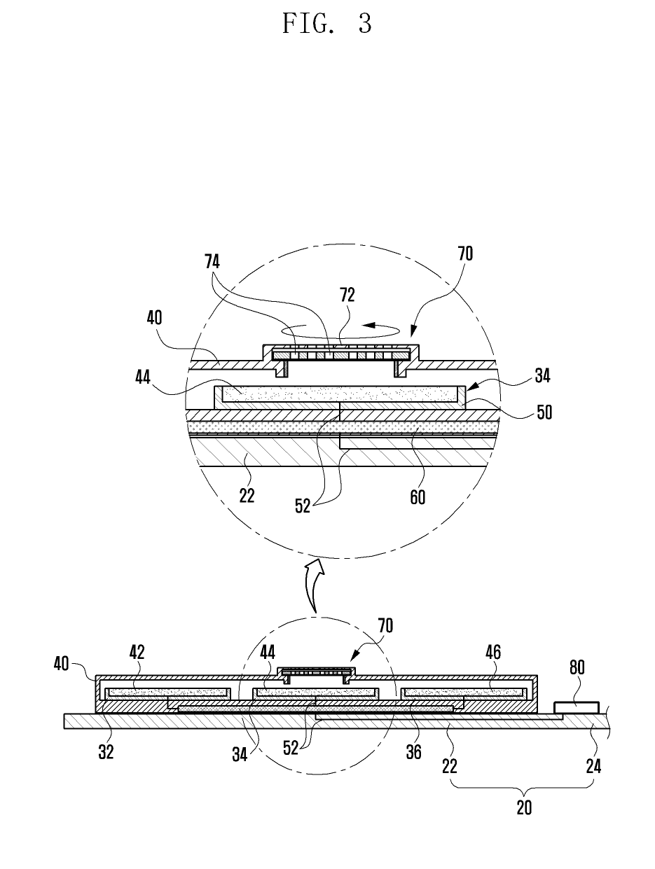

FIG. 3 is a cross-sectional view illustrating the aromatic smell generation apparatus taken along line A-A of FIG. 1.

Referring to FIG. 3, the receiving case 40 may include an exothermic pad 50 that encloses a side surface and a bottom surface of each of the different types of the plurality of aromatic smell generation cartridges 32, 34, and 36, a battery 60, and an opening and closing portion 70 which is opened and closed to refill the aromatic smell generation cartridges 32, 34, and 36. The exothermic pad 50, the battery 60, and a controller 80 are electrically connected by an interconnection mechanism 52, and operation of the exothermic pad 50 may be controlled by the controller 80.

The battery 60 is a power source of the exothermic pad and may be embedded in the receiving case 40 as a thin film type.

A stopper 72 that selectively discharges aromatic smells of perfume sticks housed in the aromatic smell generation cartridges 32, 34, and 36 may be coupled and attached to the opening and closing portion 70.

A plurality of opening portions 74 are formed in the stopper 72 and may be installed in the opening and closing portion 70 of other receiving cases 40 in order for the plurality of opening portions 74 to be selectively opened or closed according to a rotation of the stopper 72. When opening the opening portion 74 by rotating the stopper 72, an aromatic smell of the aromatic smell generation cartridges 32, 34, and 36 may be generated through the opening portion 74. In contrast, when closing the opening portion 74, an aromatic smell of the aromatic smell generation cartridges 32, 34, and 36 may not be generated through the opening portion 74.

Gel type semisolid or sticker type thin film solid perfume sticks 42, 44, and 46 containing, for example, rose aroma, lilac aroma, and herb aroma, may be housed in the aromatic smell generation cartridges 32, 34, and 36, respectively. Therefore, the perfume sticks 42, 44, and 46 may be prevented from flowing out from the aromatic smell generation cartridges 32, 34, and 36, respectively, and damaging the protection case 20 or the mobile terminal 10.

Each of the aromatic smell generation cartridges 32, 34, and 36 may be connected to the battery 60 embedded in the receiving case 40 and may be configured with other various types of perfume sticks. Gel type semisolid or sticker type thin film solid perfume sticks 42, 44, and 46 may generate an aromatic smell as a result of being heated. According to an embodiment of the present disclosure, the exothermic pad 50 is heated by the battery 60, and a side surface and a bottom surface of each of the aromatic smell generation cartridges 32, 34, and 36 are enclosed by the exothermic pad 50. Therefore, heat from the exothermic pad 50 is transferred to the gel type semisolid or sticker type thin film solid perfume sticks 42, 44, and 46 housed within the aromatic smell generation cartridges 32, 34, and 36, respectively, so that the perfume sticks 42, 44, and 46 may diffuse rose aroma, lilac aroma, and herb aroma while being heated.

According to an embodiment of the present disclosure, the exothermic pad 50 is produced with a carbon fiber printing method having no hot wires and may be heated, for example, to a maximum temperature of 52° C. within 1 minute. The controller 80 automatically detects a temperature of the exothermic pad 50 to control power consumption at a high temperature, thereby preventing overheating.

According to an embodiment of the present disclosure, the semisolid or solid perfume sticks 42, 44, and 46 may include a label, patch, paint, metal wire rod, and directional transfer film that may generate an aromatic smell such as the rose aroma, lilac aroma, and herb aroma.

The controller 80 may control the semisolid or solid perfume sticks 42, 44, and 46 enclosed with the exothermic pad 50 to generate an aromatic smell such as rose aroma, lilac aroma, and herb aroma, respectively, by being heated by the exothermic pad 50. After heating, the sticks re-harden into a semisolid or solid state, thereby stopping diffusion of the aromatic smell, by stopping the heating operation of the exothermic pad 50.

The controller 80 may control operation of the exothermic pad 50 according to opening and closing of the opening portion 74 of the stopper 72. When the opening portion 74 is opened, the controller 80 controls the heating of the exothermic pad 50 to generate aromatic smell, and when the opening portion 74 is closed, the controller 80 controls the exothermic pad 50 through cooling to not generate aromatic smell.

Further, the controller 80 may determine a residual quantity of a perfume stick within each aromatic smell generation cartridge, notify a user of the residual quantity, and refill a cartridge of a consumed perfume stick.

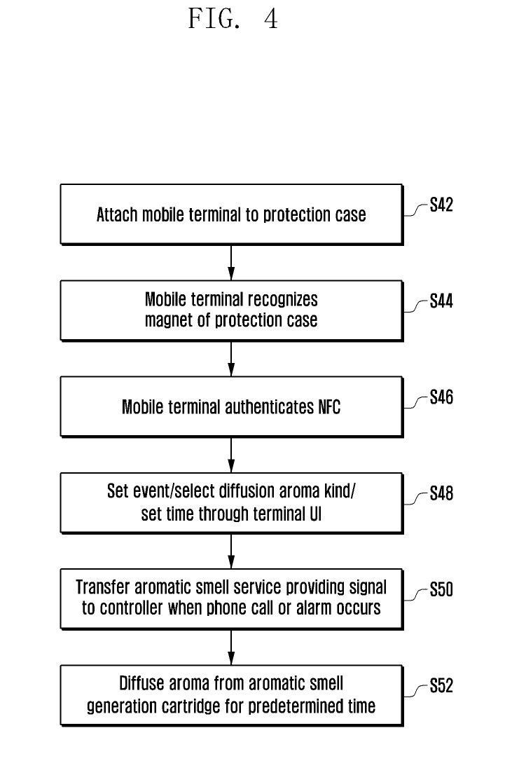

FIG. 4 is a flowchart of a method of generating aromatic smell in an electronic device, according to an embodiment of the present disclosure.

Referring to FIG. 4, in step 42, the mobile terminal 10 is attached to the protection case 20 that generates aromatic smell. In step 44, the mobile terminal 10 recognizes, based on a signal from a Hall sensor attached to the mobile terminal 10, that the mobile terminal 10 is attached to the protection case 20 through the magnet 28 of the protection case 20. In step 46, the mobile terminal 10 performs NFC authentication through the NFC IC 26 to authenticate the mobile terminal 10 that is attached to the protection case 20.

Thereafter, a phone call event, a user defined event, an alarm occurrence event, or another event may be set as an event through a UI of the mobile terminal 10 and corresponding software or firmware.

Further, when an electronic device such as a mobile terminal executes or detects an event corresponding to a designated event, diffusion of various types of aromatic smells may be undertaken to selectively generate various aromatic smells such as rose aroma, lilac aroma, and herb aroma.

Further, an aromatic smell generation time of various aromatic smells such as rose aroma, lilac aroma, and herb aroma may be set.

When the mobile terminal 10 detects an event, the aromatic smell generation apparatus 100, according to an embodiment of the present disclosure, transfers the event set to generate different aromatic smells and to diffuse the different aromatic smells to the controller 80. An event alert in the form of different aromatic smells is provided to the user of the mobile terminal in a similar method as a ringer, a vibration or a lamp flicker. The controller 80 then determines which aromatic smell to diffuse and the diffusion time. The user may set which aromatic smell to generate according to the type of event.

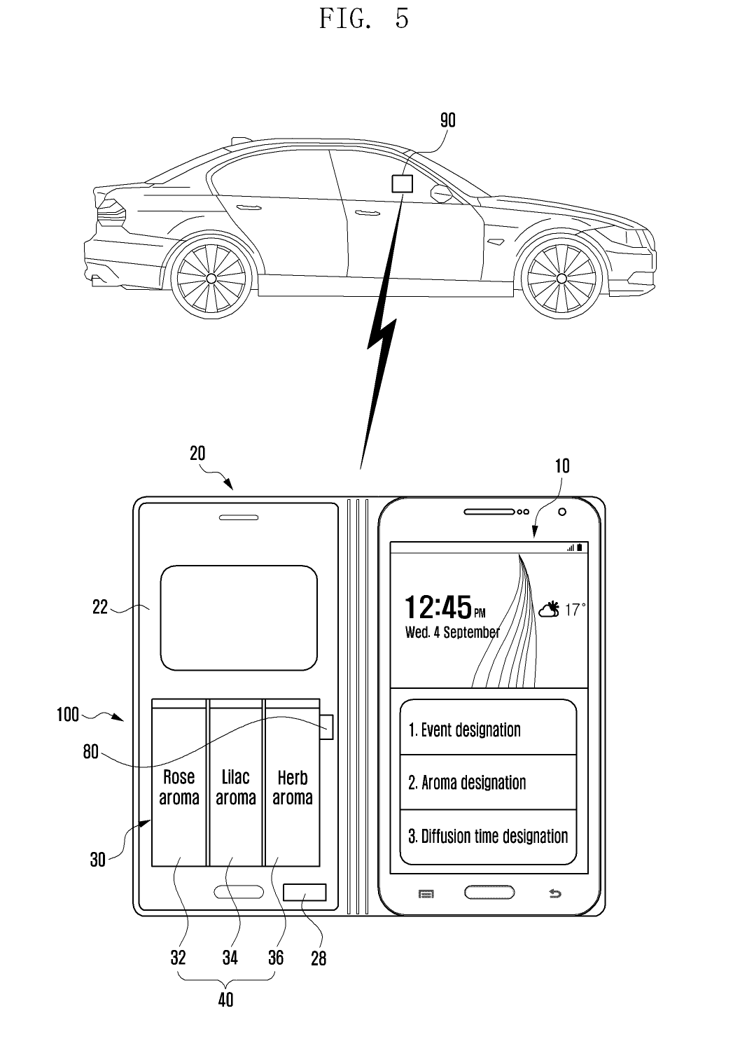

FIG. 5 is a diagram illustrating operation of an aromatic smell generation apparatus of an electronic device, corresponding to a user location, according to an embodiment of the present disclosure.

An aromatic smell generation apparatus of an electronic device according to an embodiment of the present disclosure may operate according to a user location. With reference to FIG. 5, when the user enters a vehicle, if the mobile terminal 10 receives a signal from a location tracker 90 of a mobile terminal attached to the vehicle, the mobile terminal 10 may perform NFC authentication through the NFC IC 26, and when it is determined that the user enters the vehicle, a preset aromatic smell of the aromatic smell generation cartridge may be generated. For example, when the mobile terminal user enters a specific vehicle to which the location tracker 90 is attached, the electronic device may be set such that a pleasant aromatic smell is generated from the aromatic smell generation cartridge. Various aromatic smells may be set to be generated at certain locations, as desired by the user, such as a bedroom, library, and office.

According to an embodiment of the present disclosure, various aromatic smells may be set to be generated at a location desired by the user based on a signal from a GPS receiver housed in a mobile terminal instead of a signal from a location tracker 90 of a separate mobile terminal attached to the vehicle.

As described above, according to an embodiment of the present disclosure, because a type of diffusion aroma may be selected and a diffusion time may be adjusted by a program according to an event such as an incoming call, a message, and an alarm from an electronic device, the electronic device user may be provided with a generated aromatic smell at a desired time. Further, because various types of aromatic smells may be diffused and the consumed amount, residual quantity, and replacement cycle of a diffusible aroma may be determined, an aromatic smell generation apparatus may be conveniently used when attached to an electronic device, and the functionality of the electronic device may be improved.

The embodiments disclosed in the present specification and drawings describe the technical content of the present disclosure and help the understanding of the present disclosure, but do not limit the scope of the present disclosure. Therefore, all changes or modifications derived from the technical idea of the present disclosure as well as the embodiments described herein should be interpreted to belong to the scope of the present disclosure as defined by the appended claims and their equivalents.

What is claimed is:

1. An aromatic smell generation apparatus of an electronic device comprising:
a protection case comprising a cover on one side that protects a display of the electronic device and an electronic device mounting portion on an other side in which the electronic device is located;
a cartridge attachment portion which detachably receives different types of a plurality of aromatic smell generation cartridges; and
a controller that controls a type and a diffusion time of a perfume stick within the plurality of aromatic smell generation cartridges according to a preset event.

2. The aromatic smell generation apparatus of claim 1, wherein the cover comprises:
a magnet that determines attachment and detachment of the electronic device; and
wherein the electronic device mounting portion comprises a near field communication integrated circuit (NFC IC) that determines whether the electronic device is covered by the cover.

3. The aromatic smell generation apparatus of claim 1, further comprising a receiving case that detachably receives the plurality of aromatic smell generation cartridges is attached to the cartridge attachment portion.

4. The aromatic smell generation apparatus of claim 3, wherein the receiving case comprises:
an exothermic pad that encloses a side surface and a bottom surface of each of the plurality of aromatic smell generation cartridges; and
a battery that is a power source for the exothermic pad.

5. The aromatic smell generation apparatus of claim 3, wherein the receiving case comprises an opening and closing portion to be opened and closed to attach and detach each of the plurality of aromatic smell generation cartridges.

6. The aromatic smell generation apparatus of claim 5, wherein the opening and closing portion comprises a stopper that opens and closes in order to selectively discharge aromatic smells of the perfume stick in each of the plurality of aromatic smell generation cartridges, and
wherein an aromatic smell of the plurality of aromatic smell generation cartridges is generated through an opening portion, when the opening portion is opened, and the aromatic smell of the plurality of aromatic smell generation cartridges is not generated through the opening portion, when the opening portion is closed.

7. The aromatic smell generation apparatus of claim 1, wherein gel type semisolid perfume sticks or sticker type thin film solid perfume sticks are housed in the plurality of aromatic smell generation cartridges, and the perfume sticks generate an aromatic smell when heated.

8. The aromatic smell generation apparatus of claim 4, wherein the controller detects a temperature of the exothermic pad to control power consumption and prevent the exothermic pad from being overheated.

9. The aromatic smell generation apparatus of claim 6, wherein the controller controls operation of the exothermic pad according to opening and closing of the opening portion.

10. The aromatic smell generation apparatus of claim 1, wherein the controller determines a residual quantity of the perfume stick within the plurality of aromatic smell generation cartridges and notifies a user of the residual quantity.

11. A method of generating an aromatic smell in an electronic device, the method comprising:
attaching the electronic device to a protection case that includes a plurality of aromatic smell generation cartridges that each include different types of perfume sticks;
authenticating, by the electronic device, that the electronic device is attached to the protection case;
setting, by the electronic device, a preset event; and
generating, by at least one of the plurality of aromatic smell generation cartridges, an aromatic smell according to the preset event, when the electronic device detects the preset event.

12. The method of claim 11, wherein the preset event comprises at least one of:
a phone call event, an alarm occurrence event, and a user defined event, wherein the preset event is set through a user interface (UI).

13. The method of claim 11, wherein setting the preset event comprises setting a type of aromatic smell and a diffusion time.

14. The method of claim 11, wherein the different types of perfume sticks comprise a gel type semisolid perfume stick or a sticker type thin film solid perfume stick, and the different types of perfume sticks generate the aromatic smell when heated.

15. The method of claim 11, wherein authenticating that the electronic device is attached to the protection case comprises near field communication (NFC) authentication of the electronic device.

16. The method of claim 15, wherein the protection case comprises a near field communication integrated circuit (NFC IC) for the NFC authentication.

17. The method of claim 11, wherein generating an aromatic smell according to the preset event comprises setting a type of aromatic smell and a diffusion time according to a user location.

18. The method of claim 17, wherein the user location is determined by a location tracker that transmits a user location signal.
